(19) United States
(12) Patent Application Publication
(10) Pub. No.: US 2025/0281953 A1
(43) Pub. Date: Sep. 11, 2025

(54) WASTE COMPOSTER AND WEIGHING METHOD THEREOF

(71) Applicant: Vego Garden Inc., Tomball, TX (US)

(72) Inventors: GUANG-YUAN XIONG, Tomball, TX (US); LI XIANG, Shenzhen (CN); YA-XIONG WEN, Zhongshan (CN)

(21) Appl. No.: 18/676,690

(22) Filed: May 29, 2024

(30) Foreign Application Priority Data
Mar. 6, 2024 (CN) 202410253752.2

(51) Int. Cl. B09B 3/35; B09B 101/70; G01G 19/52

(52) U.S. Cl. CPC B09B 3/35; G01G 19/52; B09B 2101/70

(57) ABSTRACT

A waste composter includes a barrel body assembly, a driving assembly, a base, and a weighing assembly. The barrel body assembly includes a barrel body and a stirring member disposed in the barrel body. The driving assembly is connected to the barrel body and the stirring member. The base includes a bottom plate and a plurality of supporting bodies which are disposed around a side of the bottom plate to form an accommodation space. The driving assembly is accommodated in the accommodation space, and the barrel body is disposed above the plurality of supporting bodies. The weighing assembly is disposed between the barrel body and the plurality of supporting bodies and is configured to weigh a total mass of the barrel body assembly, the driving assembly, and the waste in the barrel body. A weighing method of the waste composter is also disclosed.

### FIELD

The subject matter herein generally relates to the field of waste disposal, in particular to a waste composter and a weighing method thereof.

### BACKGROUND

Food waste composters are devices widely used in homes and business locations and are used to reduce a volume of food waste and convert the food waste through physical or biological methods (such as fermentation) for more environmentally friendly disposal. The design and operation of waste composters are critical to food waste management, especially in urban environments. An effective treatment of food waste can significantly reduce the total volume of the waste, reduce reliance on landfills, and potentially generate usable by-products such as compost.

However, the food waste composters currently on the market are not equipped with weighing devices, which causes several problems. Firstly, the lack of weighing devices means that users cannot know exactly how much waste has been disposed of, which is an obvious shortcoming for business locations that need to accurately calculate the cost or efficiency of waste disposal. Secondly, the lack of weighing function also affects the efficiency of biological treatment methods such as fermentation, because these methods usually require a certain amount of waste to achieve optimal treatment results. Finally, the lack of the weighing devices also prevents the user from being alerted in time when the device is full load or overloaded, which may affect the service life of the device or even cause damage to the device.

### BRIEF DESCRIPTION OF THE DRAWINGS

FIG. 1 is an overall schematic diagram of a waste composter provided by an embodiment of the present disclosure.

FIG. 2 is an exploded view of a barrel body assembly, driving assembly, base, and weighing assembly of the waste composter of FIG. 1.

FIG. 3 is a schematic diagram, from another angle of view, of the barrel body assembly, driving assembly, base, and weighing assembly of FIG. 2.

FIG. 4 is a schematic diagram of a cross section of the waste composter along a line IV-IV of FIG. 1.

FIG. 5 is a schematic diagram of a stirring member and a second connecting member of the waste composter of FIG. 1.

FIG. 6 is a is a logic control diagram of the waste composter of FIG. 1.

FIG. 7 is a schematic diagram of a position state B, a second position state C, and a third position state D provided by an embodiment of the present disclosure.

### DETAILED DESCRIPTION

The technical solutions of the present disclosure will be described in detail below with reference to the drawings and embodiments of the present disclosure. Apparently, the described exemplary embodiments are part of embodiments of the present disclosure, not all of the embodiments.

It should be noted that when an element is considered to be “disposed” on another element, the element may be directly disposed on the other element, or there may be an intermediate element.

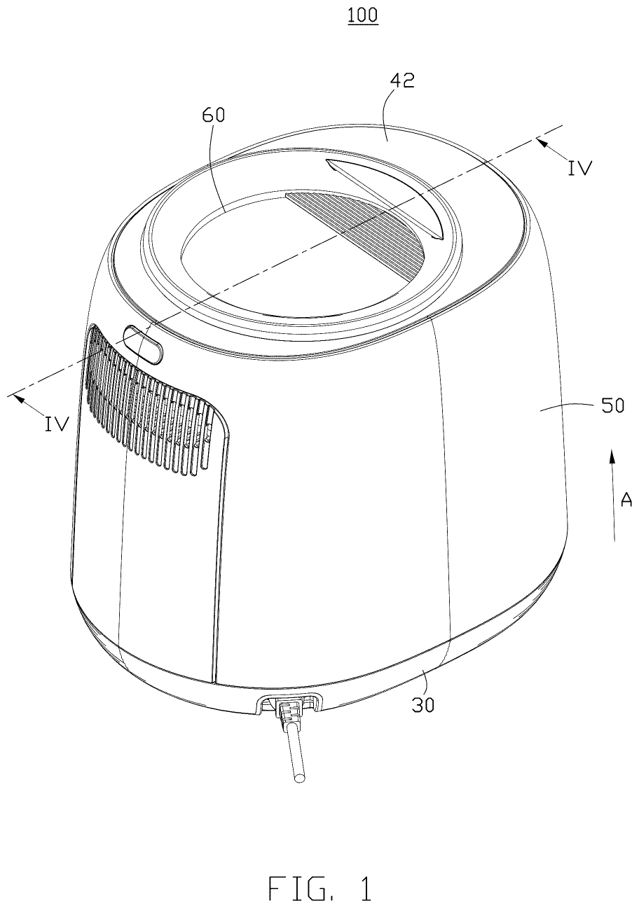

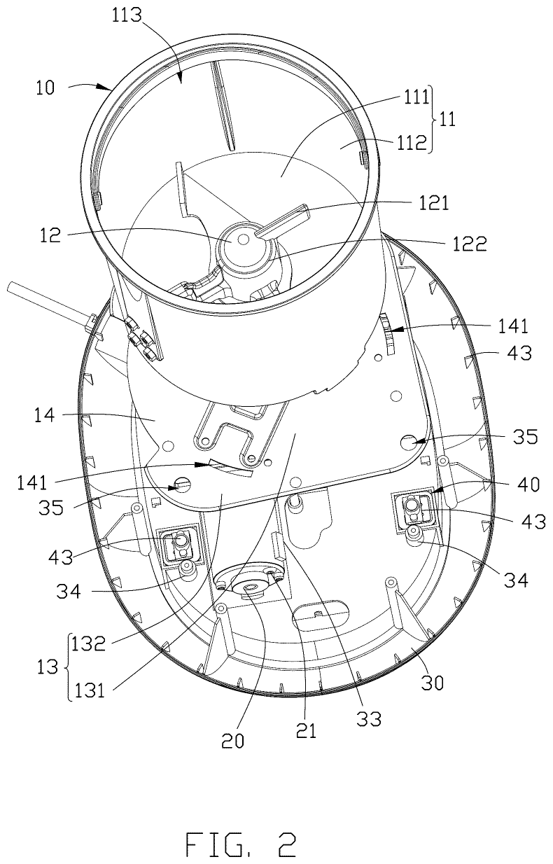

Referring to FIG. 1 and FIG. 2, a waste composter 100 of an embodiment of the present disclosure is illustrated. The waste composter 100 includes a barrel body assembly 10, a driving assembly 20, a base 30, and a weighing assembly 40. The barrel body assembly 10, the driving assembly 20, and the weighing assembly 40 are all disposed on the base 30. The driving assembly 20 is connected to an exterior of the barrel body assembly 10. The weighing assembly 40 is disposed between the barrel body assembly 10 and the base 30.

Referring to FIG. 2, the barrel body assembly 10 includes a barrel body 11 and a stirring member 12. The stirring member 12 is rotatably installed at a bottom of the barrel body 11. The barrel body 11 is used to receive waste, such as kitchen waste. The driving assembly 20 is drivingly connected to the stirring member 12 to drive the stirring member 12 to rotate, thereby stirring the waste contained in the barrel body 11.

The base 30 includes a bottom plate 31 and a plurality of supporting bodies 32. The plurality of supporting bodies 32 are arranged around one side of the bottom plate 31 to form a receiving space 33. The driving assembly 20 is located in the receiving space 33, and the barrel body 11 is disposed above the plurality of supporting bodies 32 in a vertical direction A.

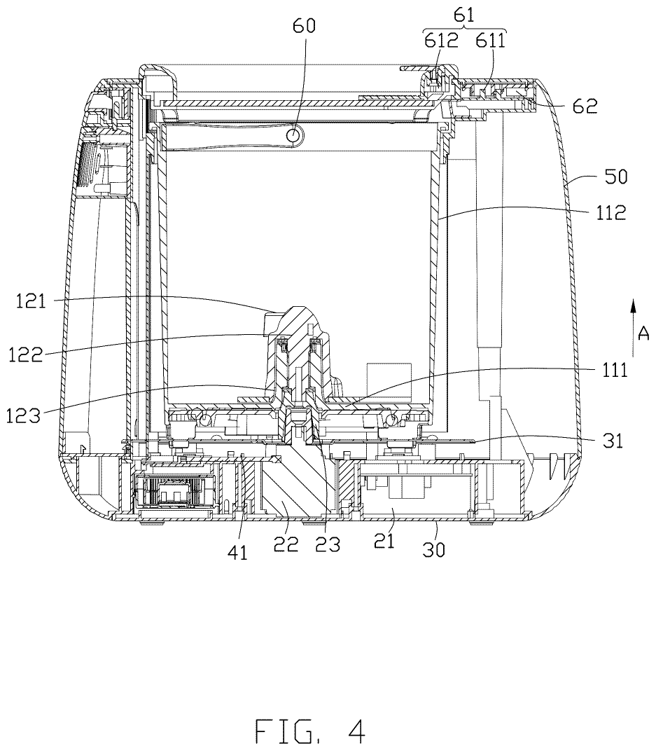

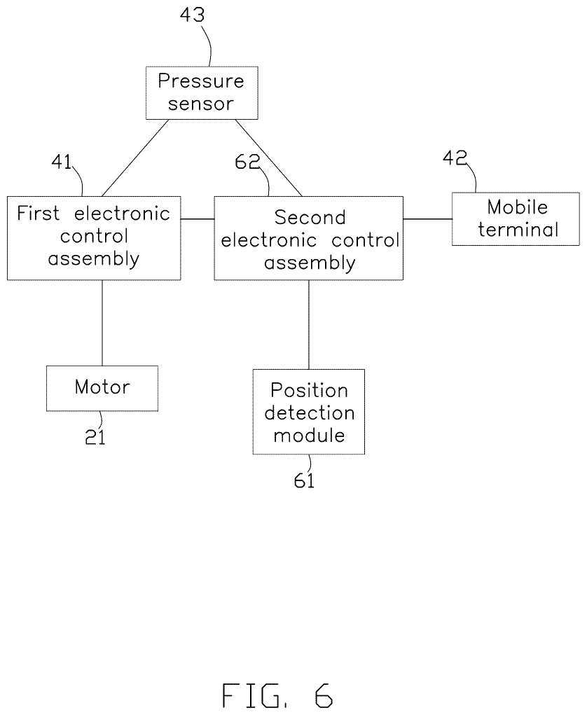

The weighing assembly 40 is disposed between each supporting body 32 and the barrel body 11. A total mass of the barrel body assembly 10 and the driving assembly 20 is defined as a first mass W1, and a total mass of the barrel body assembly 10, the driving assembly 20 and the waste contained in the barrel body 11 is defined as a second mass W2. The weighing assembly 40 is used to collect the first mass W1 and the second mass W2, and the weighing assembly 40 can obtain a mass W3 of the waste based on the difference between these two masses, that is, W3−W2−W1. A calculation process is completed through a second electronic control assembly 62 (shown in FIG. 4 and FIG. 6).

When in use, a user can adaptively adjust a stirring speed of the stirring member 12 according to the mass of the waste, thereby helping to improve processing efficiency, reducing processing time and cost, and ensuring that the waste is fully and effectively processed. Secondly, the user can better control the mass of added microorganisms based on the mass of the waste to ensure optimal conditions during a biological treatment process, thus helping to improve the efficiency of biological treatments such as fermentation and produce more useful by-products such as compost. All in all, arrangement of the waste composter 100 having a weighing function allows the user to easily understand operation status of the waste composter 100, which helps to improve the user experience and allows the user to manage and operate the waste composter more conveniently.

In the waste composter 100 provided by the present disclosure, the weighing assembly 40 is disposed between the barrel body assembly 10 and the supporting bodies 32 and is connected to the barrel body assembly 10 and the driving assembly 20, so that the weighing assembly 40 can measure the total mass of the barrel body assembly 10 and the driving assembly 20, as well as the total mass of the barrel body assembly 10, the driving assembly 20, and the waste accommodated in the barrel body assembly 10. In this way, the net weight of the waste can be obtained by weighing twice, the measurement process is more accurate, and the waste composter 100 has a simple structure.

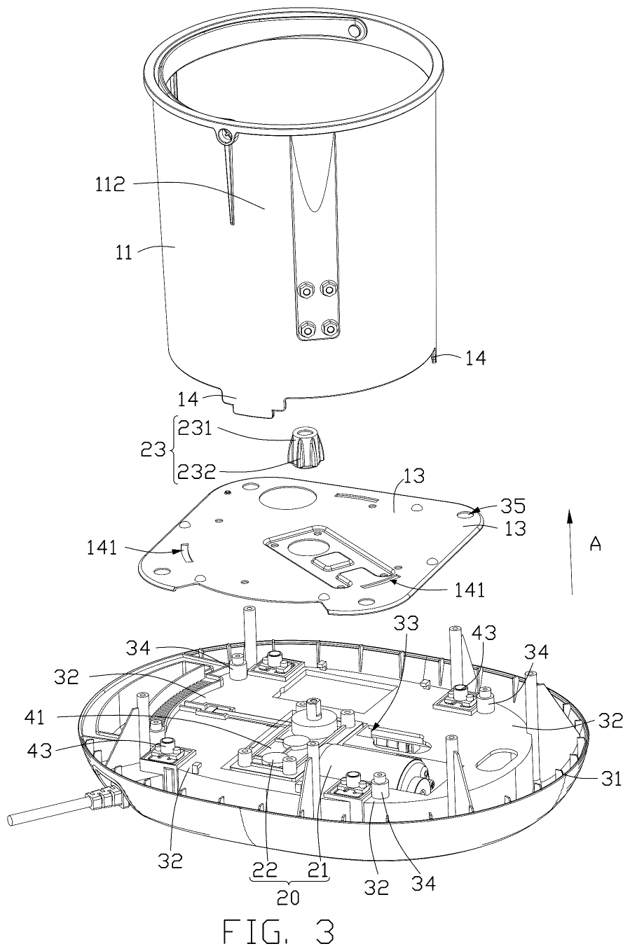

Referring to FIG. 2 and FIG. 3, in the embodiment, the barrel body assembly 10 further includes a substrate 13 and a plurality of latch portions 14. The barrel body 11 is substantially cylindric and includes a bottom plate 111 and a side plate 112. The side plate 112 is connected to the bottom plate 111 and disposed around a periphery of the bottom plate 111 to form an accommodation space 113. The accommodation space 113 is used to accommodate the waste. The bottom plate 111 extends away from the side plate 112 to form the plurality of latch portions 14. The substrate 13 is provided with a plurality of clamping grooves 141 penetrating the substrate 13 and corresponding to the plurality of latch portions 14 respectively, and each latch portion 14 is inserted into a corresponding clamping groove 141. In this way, the substrate 13 and the barrel body 11 are connected. The weighing assembly 40 is disposed between the substrate 13 and the plurality of supporting bodies 32. In other embodiments of the present disclosure, the clamping grooves 141 are defined on the bottom plate 111, and the latching portions 14 are defined on the substrate 13.

In the embodiment, the substrate 13 is substantially square and includes a main body portion 131 and a plurality of extension portions 132. The plurality of extension portions 132 protrude from an edge of the main body portion 131. Each supporting body 32 is provided corresponding to one extension portion 132. The bottom plate 111 of the barrel body 11 is disposed corresponding to the main body portion 131. The clamping groove 141 is disposed on a connection between the main body portion 131 and the extension portion 132. The weighing assembly 40 is disposed between the extension portions 132 and the supporting bodies 32. The weighing assembly 40 is used to measure a total mass W4 of the substrate 13, the latch portion 14, the barrel body 11, and the driving assembly 20, as well as a total mass W5 of the substrate 13, the latch portion 14, the barrel body 11, the driving assembly 20, and the waste in the barrel body 11. The weighing assembly 40 can obtain the mass W3 of the waste based on the difference between these two masses, that is, W3=W5−W4.

In this embodiment, the base 30 further includes a plurality of directional columns 34, and the plurality of directional columns 34 are disposed on a side of the supporting bodies 32 facing the extension portions 132. The plurality of directional columns 34 and the weighing assembly 40 are arranged in a staggered manner. The plurality of directional columns 34 extend toward the barrel body 11 in the vertical direction A. The extension portions 132 defines a plurality of directional holes 35 corresponding to the directional columns 34, respectively. Each directional column 34 can movably pass through a corresponding directional hole 35. When in use, the directional columns 34 and the directional holes 35 are used to limit the slight movement of the barrel body 11 in the vertical direction A and to avoid movement in other directions, which is beneficial to the accuracy of weighing of the weighing assembly 40.

In the embodiment, the number of the directional columns 34 is four, and the number of the directional holes 35 is four. The number of the supporting bodies 32 is four, and the number of the extending portions 132 is four. The four supporting bodies 32 are symmetrically distributed, and the four directional pillars 34 are also symmetrically distributed. In other embodiments of the present disclosure, the number of the directional columns 34, of the directional holes 35, of the supporting bodies 32, and of the extension portions 132 may be two, three, five, etc.

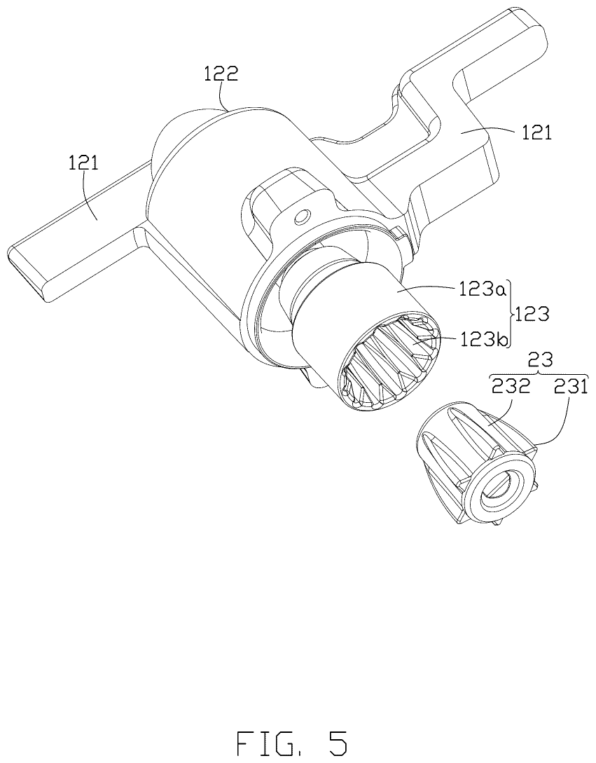

Referring to FIG. 2, FIG. 4, and FIG. 5, in the embodiment, the stirring member 12 includes a plurality of stirring paddles 121, a propeller hub 122, and a first sleeve connection member 123. One end of each of the plurality of stirring paddles 121 is connected to a periphery of the propeller hub 122. The first sleeve connection member 123 is disposed on one side of the propeller hub 122. The driving assembly 20 includes a motor 21, a speed reducer 22, and a second sleeve connection member 23. The motor 21 is connected to the speed reducer 22, and the speed reducer 22 is connected to the second sleeve connection member 23. The first sleeve connection member 123 is sleeved on the second sleeve connection member 23. In this way, the stirring member 12 is drivingly connected to the driving assembly 20. The speed reducer 22 is used to reduce the output speed of the motor 21 and to increase the output torque, so that the plurality of stirring paddles 121 can smoothly stir the waste. The speed reducer 22 is connected to the main body portion 131 through a fixing member (e.g., a screw), and the motor 21 is connected to the main body portion 131 through another fixing member (e.g., a screw). In this way, the driving assembly 20 and the barrel body assembly 10 are connected as a whole.

Referring to FIG. 5, in the embodiment, the first sleeve connection member 123 is substantially bowl-shaped, and the first sleeve connection member 123 includes a sleeve portion 123a and a plurality of protrusions 123b. The plurality of protrusions 123b extend in the vertical direction A and are disposed in the sleeve portion 123a in a spaced manner. The second sleeve member 23 includes a core shaft 231 and a plurality of grooves 232 defined on an outer surface of the core shaft 231 and spaced from each other. Each of the plurality of protrusions 123b can insert into one of the plurality of grooves 232. In this way, the first sleeve member 123 is sleeved on the second sleeve member 23.

Referring to FIG. 4 and FIG. 6, in the embodiment, the weighing assembly 40 includes a first electronic control assembly 41, a plurality of pressure sensors 43, and a second electronic control assembly 62. The weighing assembly 40 is configured to be communicatively connected to a mobile terminal 42, and the mobile terminal 42 is configured to received information transmitted by the weighing assembly 40. The second electronic control assembly 62 is electrically connected to the first electronic control assembly 41, and the plurality of pressure sensors 43 are disposed between the plurality of extension portions 132 and the plurality of supporting bodies 32 respectively. The first electronic control assembly 41 is electrically connected to the plurality of pressure sensors 43 to supply power to the plurality of pressure sensors 43. The plurality of pressure sensors 43 are electrically connected to the second electronic control assembly 62, and the second electronic control assembly 62 is configured to communicatively connected to the mobile terminal 42. The second electronic control assembly 62 is used to obtain the first mass W1 and the second mass W2 based on detection data of the plurality of the pressure sensors 43, and to calculate the mass W3 based on the first mass W1 and the second mass W2. The mobile terminal 42 is used to display the mass W3 of the waste. In other embodiments of the present disclosure, the weighing assembly 40 further includes a wireless transmission module, which is used to transmit weight information to be displayed by the mobile terminal 42 to the mobile terminal 42, such as a smartphone, etc. The number of the plurality of pressure sensors 43 is four, and each pressure sensor 43 is disposed between a corresponding supporting body 32 and a corresponding extension portion 132. The second electronic control assembly 62 is electrically connected to the four pressure sensors 43 and calculates the first mass W1 or the second mass W2 by summing mass measured by four pressure sensors 43. The second electronic control assembly 62 is one of a central processing unit, a digital signal processor, and an application-specific integrated circuit. The pressure sensors 43 are one of strain gauge pressure sensors, piezoresistive pressure sensors, and capacitive pressure sensors. It can be understood that in other embodiments of the present disclosure, the first mass W1 is stored in the second electronic control assembly 62.

In the embodiment, the first electronic control assembly 41 is further electrically connected to the motor 21 to supply power to the motor 21, and the second electronic control assembly 62 is used to control the motor 21 to be turned on or turned off through the first electronic control assembly 41. When the mass W3 of the waste calculated by the second electronic control assembly 62 is less than a minimum preset value (for example, 0.01 Kg), the second electronic control assembly 62 determines that the amount of the waste in the barrel body 11 is too small, the barrel body 11 is unloaded, and the second electronic control assembly 41 controls the first electronic control assembly 41 to stop supplying power to the motor 21, thereby achieving a purpose of reducing idling and achieving safety design. In addition, when the mass W3 of the waste calculated by the second electronic control assembly 41 is greater than a maximum preset value (for example, 10 Kg), the first electronic control assembly 41 determines the amount of the waste in the barrel body 11 is too much, the barrel body 11 is overloaded, and the second electronic control assembly 62 also controls the motor 21 to stop running, thereby reducing the problem of waste overflowing during a mixing process due to too much waste, and preventing the motor 21 from being damaged due to overload, thereby helping to extend the service life of the equipment, reduce maintenance and replacement costs, and improve the stability of the waste composter 100.

Referring to FIG. 1 and FIG. 4, in the embodiment, the waste composter 100 further includes a housing 50 and a cover body 60. The barrel body assembly 10, the driving assembly 20, and the weighing assembly 40 are all installed in the housing 50. The base 30 covers one end of the housing 50, and the cover body 60 covers the other end of the housing 50.

Referring to FIG. 1, FIG. 4, and FIG. 6, in this embodiment, the cover body 60 can abut against one end of the barrel body 11. The waste composter 100 further includes a position detection module 61 disposed on the cover body 60. The second electronic control assembly 62 is disposed on the cover body 60. The position detection module 61 is electrically connected to the first electronic control assembly 41, and the second electronic control assembly 62 is electrically connected to the position detection module 61 and the plurality of pressure sensors 43. The second electronic control assembly 62 calculates the first mass W1 or the second mass W2 by summing mass measured by the plurality of the pressure sensors 43.

The position detection module 61 is used to detect whether the cover body 60 is assembled on the barrel body 11. When the cover body 60 is assembled on the barrel body 11, the cover body 60 abuts against the barrel body 11, and the second electronic control assembly 62 controls the plurality of the pressure sensors 43 to be turned off, so that the second electronic control assembly 62 cannot collect the first mass W1 and the second mass W2, and the electronic control assembly 62 cannot send the mass W3 of the waste to the mobile terminal 41. When the cover body 60 is separated from the barrel body 11, the cover body 60 does not abut against the barrel body 11, and the second electronic control assembly 62 controls the plurality of the pressure sensors 43 to be turned on, so that the second electronic control assembly 62 can normally collect the first mass W1 and the second mass W2, and send the mass W3 of the waste to the mobile terminal 42. In this way, it is helpful to improve the accuracy of the collected first mass W1 and the second mass W2. It can be understood that if the cover body 60 abuts against the barrel body 11, measured values of the plurality of pressure sensors 43 will be greater than actual values, which may cause the mass W3 of the waste displayed by the mobile terminal 42 to be greater than an actual value of the mass of the waste. The first electronic control assembly 41 is electrically connected with the second electronic control assembly 62. The second electronic control assembly 62 includes a Bluetooth module and one of a central processing unit, a digital signal processor, or an application-specific integrated circuit.

In this embodiment, the position detection module 61 includes a Hall sensor 611 and a magnetic attraction member 612. The Hall sensor 611 is disposed on the housing 50, and the Hall sensor 611 is electrically connected to the second electronic control assembly 62. The magnetic attraction member 612 is disposed on the cover body 60, and the Hall sensor 611 can be disposed corresponding to the magnetic attraction member 612. The Hall sensor 611 is used to sense the magnetic attraction member 612. When the cover body 60 abuts against the barrel body 11, the Hall sensor 611 corresponds in position to the magnetic attraction 612, and the Hall sensor 611 senses the magnetic attraction 612 and generates a first instruction., the second electronic control assembly 62 receives the first instruction and does not collect sense data of the plurality of pressure sensors 43, so that the second electronic control assembly 62 does not send the mass W3 of the waste to the mobile terminal 42. When the cover body 60 does not abut against the barrel body 11, the Hall sensor 611 and the magnetic attraction 612 are arranged in a staggered manner, and the Hall sensor 611 does not sense the magnetic attraction 612 and generates a second instruction, the second electronic control assembly 62 receives the second instruction and collects sense data of the plurality of pressure sensors 43. In this way, the second electronic control assembly 62 sends the mass W3 of the waste to the mobile terminal 42.

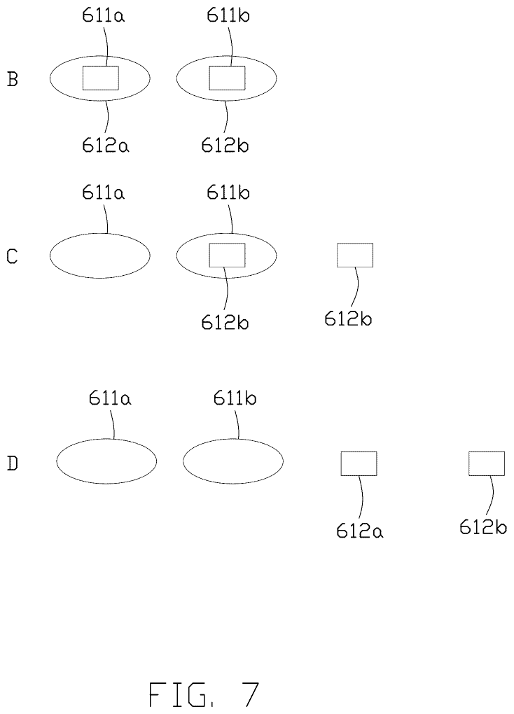

Referring to FIG. 4 and FIG. 7, in the embodiment, the cover body 60 is rotatably mounted on and covers an end of the housing 50, and the Hall sensor 611 includes a first Hall sensor 611a and a second Hall sensor 611b. The magnetic attraction member 612 includes a first magnetic attraction member 612a and a second magnetic attraction member 612b. The first Hall sensor 611a and the second Hall sensor 611b are both disposed on the housing 50 and are electrically connected to the second electronic control assembly 62. The cover body 60 and the housing 50 have a position state B, a second position state C, and a third position state D.

In the position state B, the cover body 60 is assembled on and abuts against the barrel body 11, the first Hall sensor 611a corresponds in position to the first magnetic attraction member 612a and senses the first magnetic attraction member 612a, and the second Hall sensor 611b corresponds in position to the second magnetic attraction member 612b and senses the second magnetic attraction member 612b.

When the cover body 60 rotates relative to the barrel body 11, the cover body 60 and the housing 50 are converted from the position state B to the second position state C. In the second position state C, the first Hall sensor 611a senses the second magnetic attraction member 612b, the second Hall sensor 611b is staggered with the second magnetic attraction member 612b, and the second Hall sensor 611b does not sense the second magnetic attraction member 612b.

When the cover body 60 continues to rotate relative to the barrel body 11 until the cover body 60 is separated from (i.e. no longer cover) the barrel body 11, the cover body 60 and the housing 50 are converted from the second position state C to the third position state D. In the third position state D, the cover body 60 is separated from the barrel body 11, the first Hall sensor 611a does not sense the first magnetic attraction member 612a, and the second Hall sensors 611b do not sense the second magnetic attraction member 612b.

In the position state B and the second position state C, the second electronic control assembly 62 does not collect the first mass W1 and the second mass W2 and cannot output the mass W3 of the waste to the mobile terminal 42.

In the third position state D, the second electronic control assembly 62 collects the first mass W1 of the barrel body assembly 11 and the driving assembly 20 through the plurality of pressure sensors 43, and collects the second mass W2 of the barrel body assembly 11, the driving assembly 20, and the waste contained in the barrel body 11 through the plurality of pressure sensors 43, and output the mass W3 of the waste to the mobile terminal 42.

In this way, it is possible to avoid calculating the mass of the waste when the cover body 60 is not separated from the barrel body 11 or is not completely separated from the barrel body 11, which is beneficial to improving the accuracy of waste weighing.

An embodiment of the present disclosure further provides a weighing method for the waste composter 100 as described above. The weighing method includes the following steps.

Step S1, the cover body is removed.

Step S2, the second electronic control assembly 62 collects the first mass W1 of the barrel body assembly 11 and the driving assembly 20 through the plurality of pressure sensors 43.

Step S3, the second electronic control assembly 62 collects the second mass W2 of the barrel body assembly 11, the driving assembly 20, and the waste contained in the barrel body 11 through the plurality of pressure sensors 43.

Step S4, the second electronic control assembly 62 obtains the mass W3 of the waste based on the difference between the two total masses.

In the embodiment, the cover body 60 is rotatably mounted on one end of the housing 50, and the position detection module 61 includes the first Hall sensor 611a, the second Hall sensor 611b, the first magnetic attraction member 612a and the second magnetic attraction member 612b. The first Hall sensor 611a and the second Hall sensor 611b are both disposed on the housing 50 and electrically connected to the second electronic control assembly 62. The cover body 60 and the housing 50 have the position state B, the second position state C, and the third position state D. Step S1 specifically includes the following step.

Step S11, the cover body 60 is rotated, so that the cover body 60 and the housing 50 is changed from the position state B or the second position state C to the third position state D.

In the embodiment, the weighing method further includes the following step between step S1 and step S2.

Step S12, wait for a preset time, which is 0.5 to 1 second, so that the barrel body 11 is in a balanced and static state. In this way, it is helpful to reduce inaccuracy of weighing caused by vibration of the barrel body 11 due to the rotation of the cover body 60, and further improve the accuracy of weighing.

Those of ordinary skill in the art should realize that the above embodiments are only used to illustrate the present disclosure and are not used to limit the present disclosure. As long as they are within the scope of the essential spirit of the present disclosure, appropriate changes can be made to the above embodiments. and changes all fall within the scope disclosed in this disclosure.

What is claimed is:

1. A waste composter, comprising:
a barrel body assembly, the barrel body assembly comprising a barrel body and a stirring member disposed in the barrel body, the barrel body being configured to contain waste;
a driving assembly, the driving assembly being connected to an exterior of the barrel body, the driving assembly being drivingly connected to the stirring member to drive the stirring member to stir the waste;
a base, the base comprising a bottom plate and a plurality of supporting bodies, the plurality of supporting bodies being disposed around a side of the bottom plate to form an accommodation space, wherein the driving assembly is accommodated in the accommodation space, and the barrel body is disposed above the plurality of supporting bodies in a vertical direction; and
a weighing assembly, the weighing assembly being disposed between the barrel body and the plurality of supporting bodies, the weighing assembly being configured to weigh a total mass of the barrel body assembly and the driving assembly or to weigh a total mass of the barrel body assembly, the driving assembly, and the waste in the barrel body.

2. The waste composter of claim 1, wherein the barrel body assembly further comprises a substrate and a plurality of latch portions, the plurality of latching portions protrudes from a side of the barrel body facing toward the base, the substrate defines a plurality of clamping grooves corresponding to the plurality of latching portions respectively, each of the plurality of latching portions is inserted into a corresponding clamping groove of the plurality of clamping grooves, the weighing assembly is disposed between the substrate and the plurality of supporting bodies.

3. The waste composter of claim 2, wherein the substrate comprises a main body portion and a plurality of extension portions protruding from an edge of the main body portion, each of the plurality of supporting bodies is disposed corresponding to a corresponding extension portion of the plurality of extension portions, the weighing assembly is disposed between the plurality of extension portions and the plurality of support bodies.

4. The waste composter of claim 3, wherein the base further comprises a plurality of directional columns, the plurality of directional columns is disposed on sides of the plurality of supporting bodies facing the plurality of extension portions respectively, the plurality of directional columns is staggered with the weighing assembly, the plurality of directional columns extends towards the barrel body along the vertical direction, the plurality of extension portions defines a plurality of directional holes corresponding to the plurality of directional columns respectively, each of the plurality of directional columns is adapted to movably pass through a corresponding directional hole of the plurality of directional holes.

5. The waste composter of claim 1, wherein the stirring member comprises a first sleeve connection member, a propeller hub, and a plurality of stirring paddles, an end of each of the plurality of stirring paddles is connected to a periphery of the propeller hub, the first sleeve connection member is disposed on a side of the propeller hub, the driving assembly comprises a motor, a speed reducer, and a second sleeve connection member, the first sleeve connection member is sleeved on the second sleeve connection member; the first sleeve connection member comprises a sleeve portion and a plurality of protrusions disposed on the sleeve portion in a spaced manner, the second sleeve connection member comprises a core shaft and a plurality of grooves defined on an outer surface of the core shaft, each of the plurality of protrusions is configured to insert into a corresponding groove of the plurality of grooves, the motor is driving connected to the speed reducer, and the speed reducer is connected to the second sleeve connection member.

6. The waste composter of claim 5, wherein the weighing assembly comprises a first electronic control assembly and a plurality of pressure sensors, the plurality of pressure sensors are disposed between the barrel body and the plurality of supporting bodies respectively, the plurality of pressure sensors is electrically connected to the first electronic control assembly, the plurality of pressure sensors is configured to weigh the total mass of the barrel body assembly, the driving assembly, and the waste in the barrel body, the first electronic control assembly is configured to supply power to the motor and the plurality of pressure sensor.

7. The waste composter of claim 6, further comprising a housing and a cover body, wherein the barrel body assembly, the driving assembly, and the weighing assembly are all disposed in the housing, the base covers an end of the housing, and the cover body covers another end of the housing;
the cover body is adapted to abut against an end of the barrel body, the waste composter further comprises a position detection module and a second electronic control assembly electrically connected to the position detection module, the position detection module is disposed between the cover body and the housing, the second electronic control assembly is connected to the housing, the second electronic control assembly is electrically connected to the plurality of pressure sensors and the first electronic control assembly, the second electronic control assembly is configured to calculate the mass of the waste based on the total mass of the barrel body assembly, the driving assembly, and the waste in the barrel body, the position detection module is configured to detect whether the cover body abuts against the barrel body.

8. The waste composter of claim 7, wherein the position detection module comprises a Hall sensor and a magnetic attraction member, the Hall sensor is disposed on the housing and is electrically connected to the second electronic control assembly, the magnetic attraction member is disposed on the cover body, the Hall sensor is disposed corresponding to the magnetic attraction member; when the cover body is assembled on the barrel body, the Hall sensor senses the magnetic attraction member, and the second electronic control assembly is configured to not collect signals of the plurality of pressure sensors; when the cover body is not assembled on the barrel, the Hall sensor does not sense the magnetic attraction, and the second electronic control assembly is configured to collect the signals of the plurality of pressure sensors.

9. A weighing method of the waste composter of claim 8, wherein the total mass of the of the barrel body assembly and the driving assembly is defined as a first mass, the total mass of the barrel body assembly, the driving assembly, and the waste in the barrel body is defined as a second mass, the weighing method comprises steps of:
collecting the first mass and the second mass through a plurality of the weighing assemblies when the Hall sensor does not sense the magnetic attraction member;
obtaining the mass of the waste based on a difference between the first mass and the second mass.

10. The weighing method of claim 9, wherein the first mass is stored in the second electronic control assembly, the cover body is rotatably mounted on the another end of the housing, the position detection module comprises a first Hall sensor, a second Hall sensor, a first magnetic attraction member, and a second magnetic attraction member, the first Hall sensor and the second Hall sensor are both disposed on the housing and electrically connected to the second electronic control assembly, the first magnetic attraction member and the second attraction member are both disposed on the cover body, the cover body has a position state relative to the housing, in the position state, the cover body is separated from the barrel body, the first Hall sensor does not sense the first magnetic member, the second Hall sensor does not sense the second magnetic member, the weighing method further comprises steps of:
collecting the second mass, when in the position state, using the second electronic control assembly, through the plurality of pressure sensors;
obtaining the mass of the waste, using the second electronic control assembly, based on the difference between the first mass and the second mass.
